(19) United States
(12) Patent Application Publication
(10) Pub. No.: US 2023/0263653 A1
(43) Pub. Date: Aug. 24, 2023

(54) AN OSTOMY APPLIANCE

(71) Applicant: SALTS HEALTHCARE LIMITED, Birmingham West Midlands (GB)

(72) Inventors: Jesus Alfaro, Birmingham West Midlands (GB); Lee Howard, Birmingham West Midlands (GB)

(73) Assignee: SALTS HEALTHCARE LIMITED, Birmingham West Midlands (GB)

(21) Appl. No.: 18/024,410

(22) PCT Filed: Aug. 31, 2021
(86) PCT No.: PCT/GB2021/052244

§ 371 Date: Mar. 2, 2023

(30) Foreign Application Priority Data
Sep. 3, 2020 (GB) 2013883.0

(51) Int. Cl. A61F 5/445; A61F 5/441; A61F 5/44

(52) U.S. Cl. CPC A61F 5/445; A61F 5/441; A61F 5/4404

(57) ABSTRACT

An ostomy Appliance (10) including first and second walls (12, 14) connected to each other at or near their peripheries, a stoma-receiving opening (16), a collecting cavity (18) defined between the first and second walls wherein the stoma-receiving opening is fluidly connected to the collecting cavity, a connection member (20) connected to the first wall for attaching the appliance directly or indirectly to a user, a gas vent (8) for venting gas from the collecting cavity to atmosphere, a filter (9) positioned over the gas vent, said filter defining a first discrete area, a third wall (30), wherein the second wall includes a first surface (34) and the third wall includes a second surface (56), wherein the first and second surfaces face each other and are connected at a connection (60), and wherein the connection defines a second discrete area.

### BACKGROUND

Ostomy appliances are well known. Typically ostomy appliances have first and second walls which are connected at their peripheries to form a cavity for the collection of waste. The first or second wall is provided with a stoma-receiving opening for receipt of a user's stoma. The stoma-receiving opening is typically surrounded by a connection member such as a flange or an adhesive wafer (typically formed of hydrocolloid). A common issue with ostomy appliances is that of “pancaking”. This can be caused by waste entering the cavity and adhering to both the first and second walls. This then adheres the first and second walls to each other and prevents further waste from readily entering the cavity and falling to a bottom thereof. This issue may also be generated by static charge or adhesive forces between the first and second walls, and/or the presence of a relative vacuum in the cavity, which result in the first and second walls adhering to each other and preventing waste from entering the cavity. In the most severe cases waste which cannot enter the cavity and fall to a bottom thereof may cause detachment of the connection member from a user. This is clearly undesirable and a source of embarrassment for a user.

The present invention seeks to address these problems.

According to a first aspect of the invention we provide an ostomy appliance including:
first and second walls connected to each other at or near their peripheries;
a stoma-receiving opening for receipt of a stoma;
a collecting cavity defined between the first and second walls wherein the stoma-receiving opening is fluidly connected to the collecting cavity;
a connection member connected to the first wall for attaching the appliance to a user or for attaching the appliance to a flange for attaching the appliance to a user;
a gas vent for venting gas from the collecting cavity to atmosphere;
a filter positioned over the gas vent, said filter defining a first discrete area;
a third wall optionally connected to the first and/or second wall at or near its periphery;
wherein the second wall includes a first surface and the third wall includes a second surface;
wherein the first and second surfaces face each other and are connected at a connection; and
wherein the connection defines a second discrete area.

Movement of the third wall away from the first wall may at least partially separate the first and second walls so as to permit air to flow through the gas vent into the collecting cavity.

The third wall may overlie the filter.

According to a second aspect of the invention we provide an ostomy appliance including:
first and second walls connected to each other at or near their peripheries;
a stoma-receiving opening for receipt of a stoma;
a collecting cavity defined between the first and second walls wherein the stoma-receiving opening is fluidly connected to the collecting cavity;
a connection member connected to the first wall for attaching the appliance to a user or for attaching the appliance to a flange for attaching the appliance to a user;
a gas vent provided in the first wall for venting gas from the collecting cavity to atmosphere;
a filter positioned over the gas vent; and
a third wall connected to the second wall at a connection;
wherein movement of the third wall in a direction away from the first wall at least partially separates the first and second walls and permits air to flow through the gas vent into the collecting cavity.

The filter may define a first discrete area.

The connection may define a second discrete area.

The third wall may be or may include a comfort layer.

The ostomy appliance may include a plurality of connections between the second and third walls.

The connection or at least one of the connections between the second and third walls may be an adhesive connection.

The connection or at least one of the connections between second and third walls may be provided by a weld.

The connection or at least one of the connections may be positioned vertically below the filter, in an in use position of the appliance.

The connection or at least one of the connections may be positioned completely or at least partially above a generally horizontal line which extends through the stoma-receiving opening.

The connection or at least one of the connections may have a generally elongate axis which is inclined to a generally vertical axis of the ostomy appliance.

The connection or at least one of the connections may have a generally elongate axis which intersects the filter and/or gas vent.

The ostomy appliance may include first and second connections between the second and third walls. The first and second connections may be mirror images of each other.

The third wall may be positioned in an upper portion of the appliance. The third wall may be connected to the second wall of the appliance.

The third wall may cover a top portion of the appliance. The third wall may extend downwardly at least past a main horizontal axis of the stoma-receiving opening.

One or more or all of the features of the second aspect of the invention may be incorporated into the first aspect of the invention.

One or more or all of the features of the first aspect of the invention may be incorporated into the second aspect of the invention.

Embodiments of the invention will now be described by way of example only with reference to the accompanying drawings, of which:

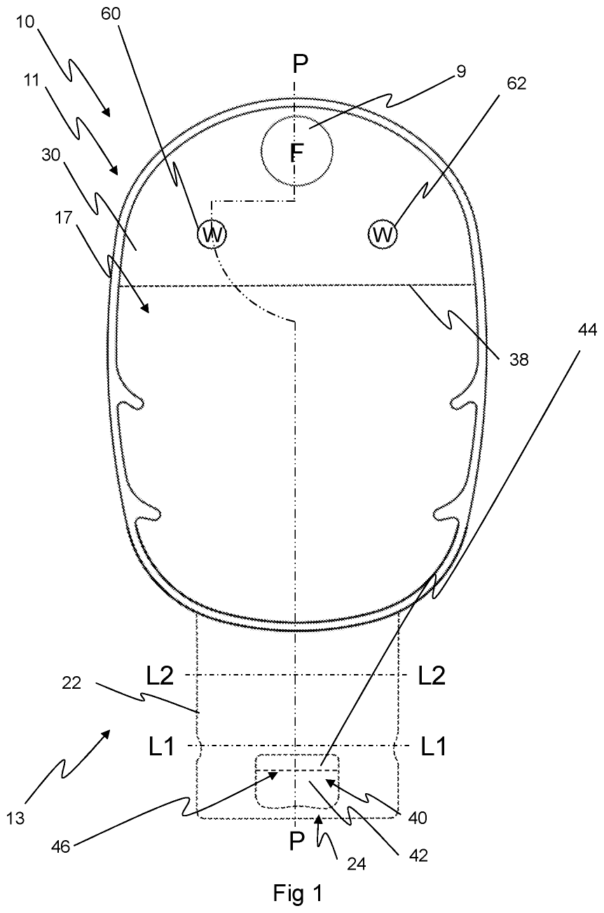

FIG. 1 is a front view of a first embodiment of an ostomy appliance in accordance with the present invention;

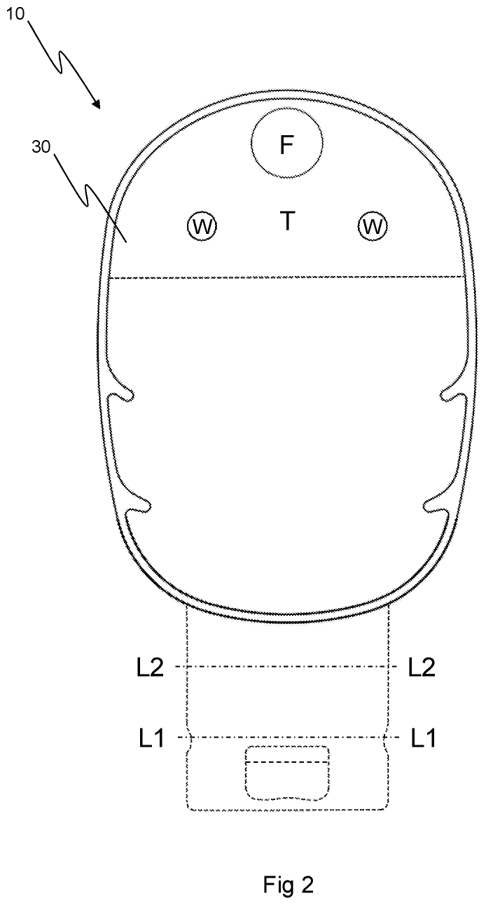

FIG. 2 is a further front view of the ostomy appliance of FIG. 1;

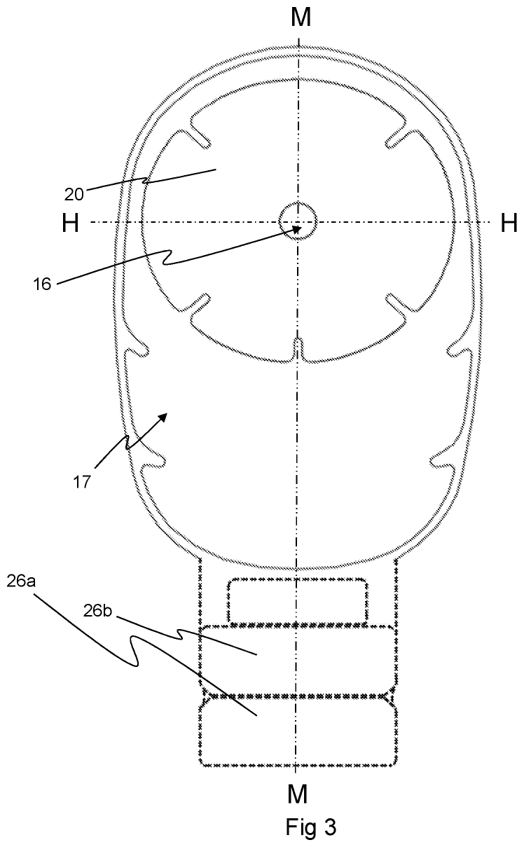

FIG. 3 is a rear view of the ostomy appliance of FIG. 1;

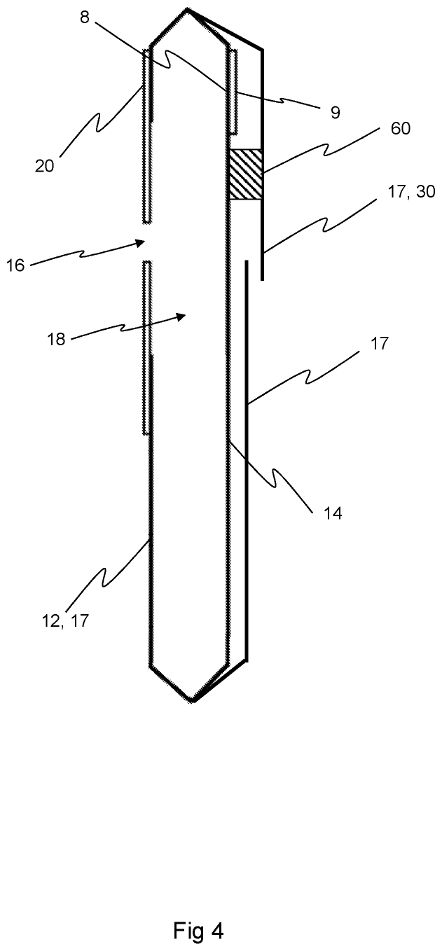

FIG. 4 is a cross-sectional view of the ostomy appliance of FIG. 1 looking at the section of the ostomy appliance indicated by the line P;

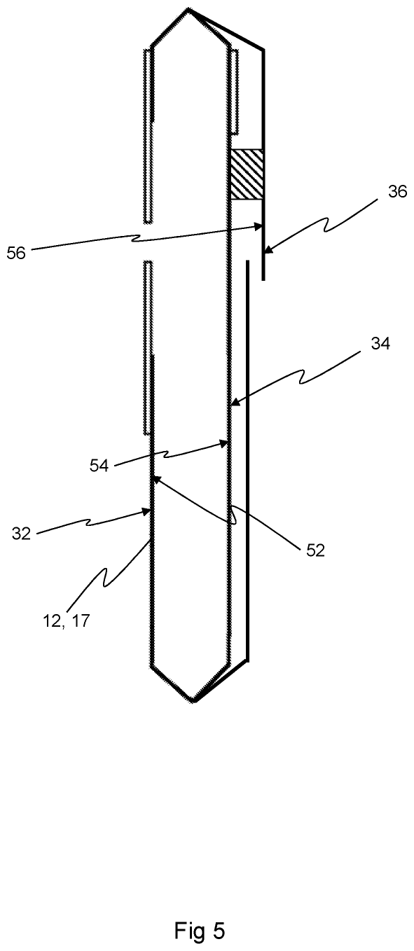

FIG. 5 is a further cross-sectional view of the ostomy appliance of FIG. 1 looking at the section of the ostomy appliance indicated by the line P;

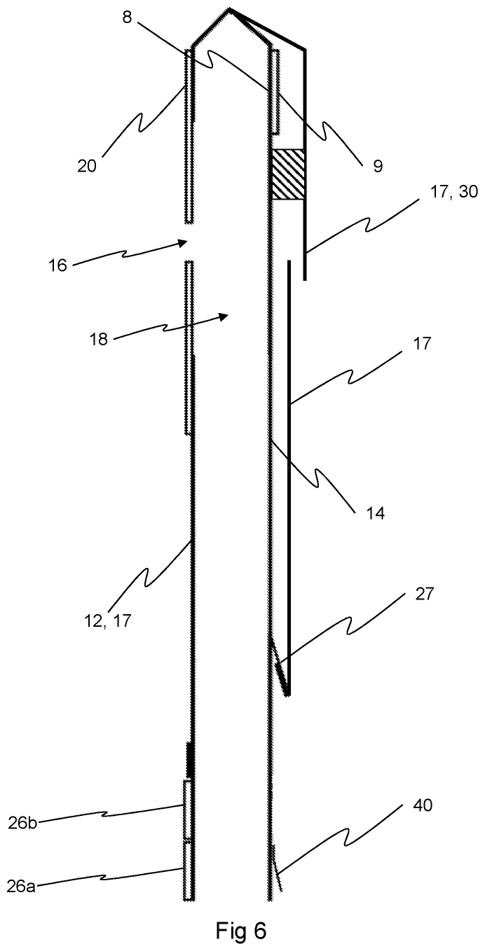

FIG. 6 is a further cross-sectional view of the ostomy appliance of FIG. 1 looking at the section of the ostomy appliance indicated by the line P with the section shown in dashed lines in FIG. 1 shown in solid lines;

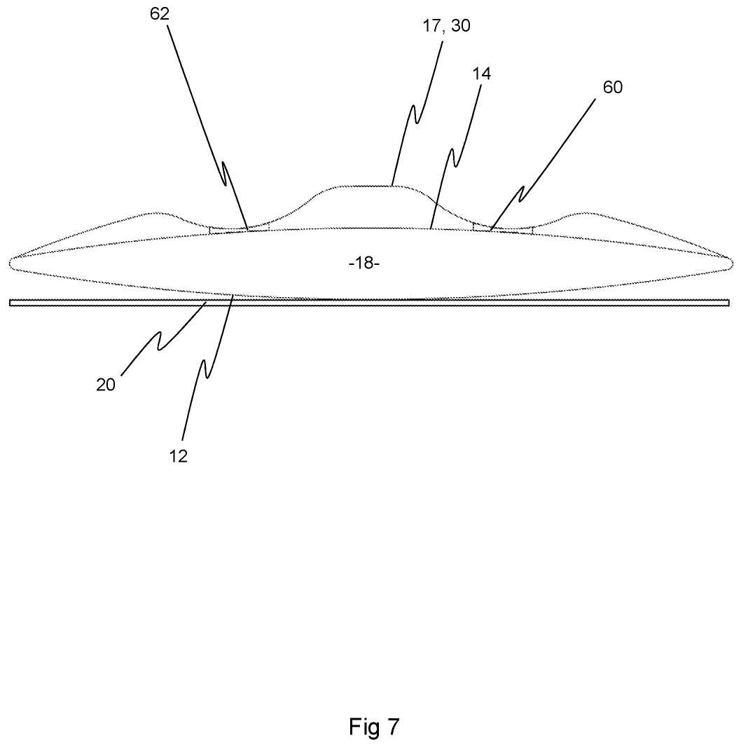

FIG. 7 is a top view of the ostomy appliance of FIG. 1 with the first, second and third walls adjacent each other in a partially open position;

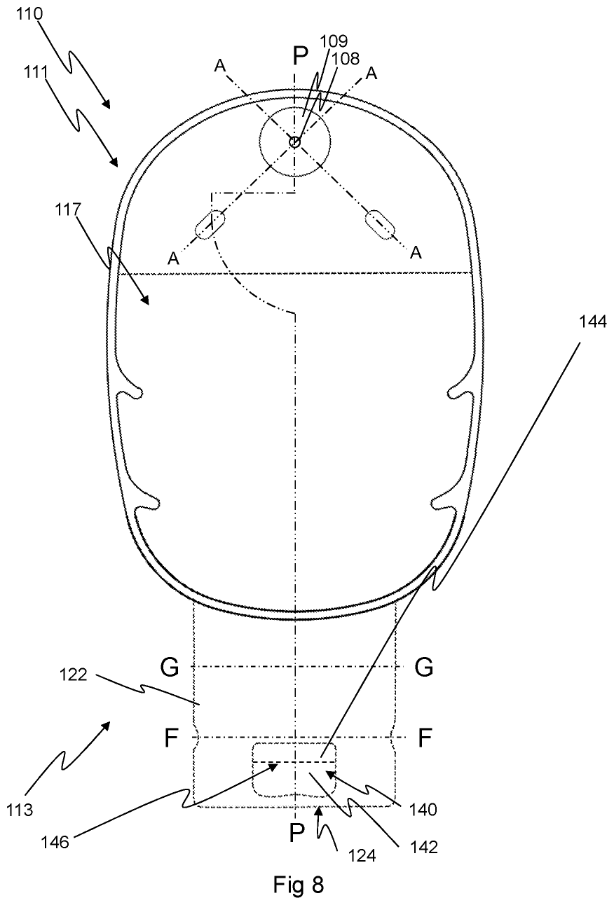

FIG. 8 is a front view of a second embodiment of an ostomy appliance in accordance with the present invention;

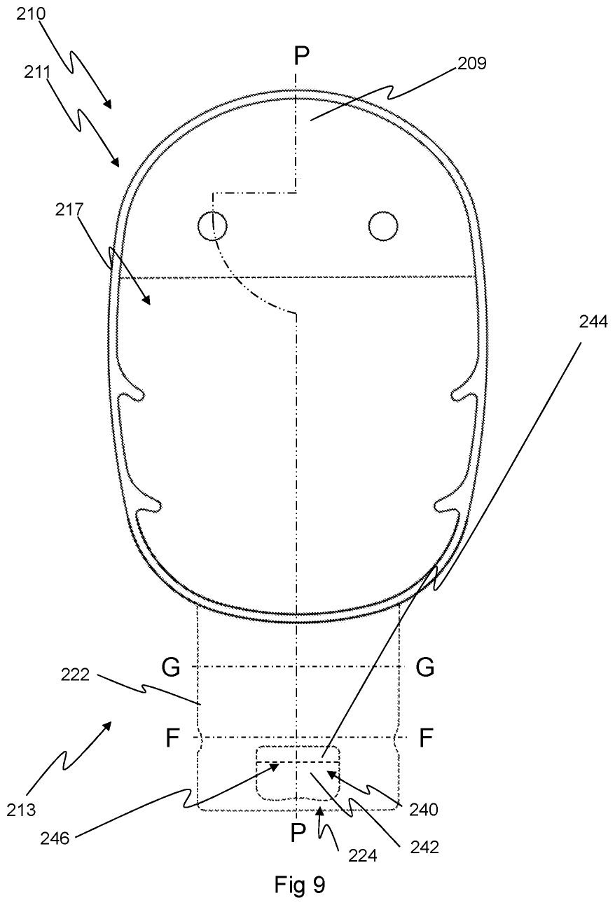

FIG. 9 is a front view of a third embodiment of an ostomy appliance in accordance with the present invention; and

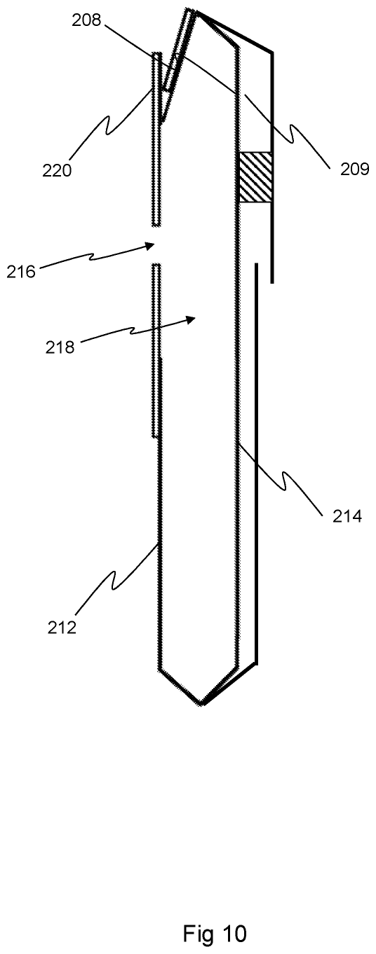

FIG. 10 is a cross-sectional view of the ostomy appliance of FIG. 9 looking at the section of the ostomy appliance indicated by the ostomy appliance indicated by the line P.

Referring to FIGS. 1 to 7, these show a first embodiment of an ostomy appliance in accordance with the present invention, generally at 10. The ostomy appliance 10 has a top, or upper end, 11 and a bottom, or lower end, 13. The ostomy appliance 10 has first and second walls 12, 14 (preferably made from a plastics film material, which may be multi-layer) connected to each other at or near their peripheries, by any suitable means known in the art. For example, they could be adhered to each other or heat welded.

The first wall 12 includes a stoma-receiving opening 16 and supports a connection member or flange 20 for attaching the appliance 10 to a user. In the embodiment shown, the connection member 20 is a hydrocolloid wafer for securing the appliance 10 to the skin of a user around their stoma. Alternatively, the connection member 20 may be for attaching the appliance 10 to a flange for attaching the appliance to a user (e.g. a two-piece appliance as it is known in the art).

Referring to FIG. 4, the first and second walls 12, 14 define a waste collecting cavity 18 therebetween which is fluidly connected to the stoma-receiving opening 16. In the present embodiment the first and second walls 12, 14 include, first, respectively outwardly facing surfaces 32, 34. The first and second walls 12, 14 also include, second, respectively inwardly facing surfaces 52, 54. The first and second walls 12, 14, and in particular the outwardly facing surfaces 32, 34, are covered by comfort layers 17 though these are not essential. For example only the first or second wall 12, 14 may be provided with a comfort layer, or neither the first nor the second wall 12, 14 may be provided with a comfort layer. The comfort layers 17 may be laminated to the first and/or second walls 12, 14.

The appliance 10 includes a filter 9 positioned over a suitable gas vent 8 in the second 14 wall, as is well known in the art. In particular, the filter 9 is positioned on the first, outwardly facing, surface 34 of the second wall 44 (though in other embodiments the filter 9 may be positioned on the second, inwardly facing, surface 34 of the second wall 14). In other embodiments the filter 9 may be positioned over a suitable gas vent in the first wall 12 (on either the first or second surfaces 32, 52 of the first wall 12). In further envisaged embodiments a plurality of gas vents 8 may be provided in the first and/or second walls 12, 14. Filters 9 may be positioned over the respective gas vents 8 in the first and/or second walls 12, 14 (on the first and/or second respective surfaces 32, 34, 52, 54). The filter 9, or each filter in those embodiments where there are a plurality of filters, defines a first discrete area F.

A further third wall 30 at least partially overlies the second wall 14. The third wall 30 may also overlie the or each filter 9 but this need not necessarily be the case. The third wall 30 is optionally connected to the first and/or second walls 12, 14. In particular the third wall 30 may be connected to the first and/or second walls 12, 14 at or near its peripheries except for a bottom edge 38 thereof. In the present embodiment the third wall 30 is positioned in an upper portion 11 of the ostomy appliance 10 and is connected to the second wall 14 of the ostomy appliance 10. The third wall 30 covers a top portion of the appliance 10 and extends downwardly at least past a main horizontal axis of the stoma-receiving opening 16. The third wall 30 may be made from a plastics film material, which may be multi-layer. Alternatively the third wall 30 may be made from other materials. For example, in the present embodiment the third wall 30 is formed by a portion of the comfort layer 17 which covers the second wall 14. The third wall 30 in the present embodiment also includes a plastics film material which the comfort layer 17 overlies at least partially. In particular a first, outwardly facing, surface 36 of the third wall 30 is attached to a portion of the comfort layer 17 by a lamination process.

As explained above the third wall 30 has a first, outwardly facing, surface 36. The first, outwardly facing, surface 36 faces away from a user and the first and second walls 12, 14. The third wall 30 also includes a second, inwardly facing, surface 56 which faces towards the user and the first and second walls 12, 14. In particular, the second surface 56 of the third wall 30 and the first surface 34 of the second wall 14 face each other and are connected at a connection 60. The connection 60 defines a second discrete area W. The connection 60 is described in more detail later.

The third wall 30 may cover a third area T of the ostomy appliance 10. The first and second areas F and W may lie within the third area T. In other embodiments only the second area W may lie within the third area T.

The third wall 30 is movable in a direction away from the first wall 12. The third wall 30 is also movable in a direction away from the second wall 14. The third wall 30 is also movable in a direction generally away from a user. Movement of the third wall 30 away from the first wall 12 at least partially separates the first and second walls 12, 14, as shown at FIG. 5. In particular movement of the third wall 30 causes movement of the connection 60 away from the first wall 12. This in turn causes movement of the second wall 14 away from the first wall 12. Movement of the third wall 30 in this manner permits air to flow through the gas vent 8 into the collecting cavity 18.

Such a construction is advantageous. By moving the third wall as described above a user is able to solve the issue of pancaking by overcoming the adhesive force caused by waste sticking the first and second walls together. Such movement of the third wall also assists in overcoming pancaking which occurs due to vacuum/static forces between the first and second walls 12, 14. By moving the third wall 30 the second wall 14 is moved away from the first wall 12 so as to overcome any static forces between the first and second walls 12, 14. In other words, movement of the third wall away from the first wall 12 increases the volume of the waste collecting cavity 18 which creates a greater vacuum, or pressure differential between the waste collecting cavity 18, a user and atmosphere. To resolve the pressure difference either air will enter the ostomy appliance 10 through the gas vent 8 and thus assist in maintaining the first and second walls 12, 14 in a spaced apart configuration or waste will enter through the stoma-receiving opening 16 and enter the waste collecting cavity 18. This waste will then maintain the first and second walls 12, 14 in a spaced apart configuration. Therefore, the invention enables a user to easily remove the issue of pancaking and continue to use the ostomy appliance without the need to replace the ostomy appliance.

In the present embodiment first and second connections 60, 62 are provided between the second and third walls 14, 30. However, in other embodiments only a single connection 60 may be provided. In further envisaged embodiments a plurality of connections 60 may be provided between the second and third walls 14, 30. The connections 60, 62 are generally the same shape. In other embodiments the connections 60 may be different shapes.

In the present embodiment the first and second connections 60, 62 are generally circular. However, the first and second connections 60, 62 may be other shapes without departing from the scope of the present invention. For example, the connections 60 may be elliptical, obround/racetrack shaped, triangular or any other polygonal shape without departing from the scope of the present invention.

The or each connection 60, 62 may be provided by an adhesive connection between the second and third walls 14, 30. In addition the or each connection 60, 62 may be provided by a weld between the second and third walls 14, 30.

The connection, or at least one of the connections, 60, 62 is positioned vertically below the filter 9, in an in use position of the ostomy appliance 10. By vertically we mean at a position below the filter 9 on a midline M which extends generally between the top 11 and bottom 13 of the appliance 10 which is generally perpendicular to a horizontal line H which extends through the stoma-receiving opening 16 and a main axis of the stoma-receiving opening 16 through which a stoma extends in use. In other words, we mean that the connections 60, 62 are not necessarily directly beneath the filter 9, but are positioned vertically closer to the bottom 13 of the ostomy appliance 10 than the filter 9. In some embodiments the or each connection 60 may be provided directly below the filter 9. The connection, or at least one of the connections 60, 62 may be positioned vertically above the stoma-receiving opening 16. In particular, the connection, or at least one of the connections, 60, 62 is positioned completely or at least partially above the generally horizontal line H which extends through the stoma-receiving opening 16. In embodiments, the connections 60, 62 may be positioned at a periphery of a maximum size to which the stoma-receiving opening 16 may be enlarged.

The first and second connections 60, 62 lie either side of the filter 9 generally opposite each other. In particular, the first and second connections 60, 62 are mirror images of each other.

In the present embodiment the first and second connections 60, 62 are separate from the peripheral connection between the first, second and third walls 12, 14, 30. However, in other embodiments the connection(s) 60, 62 may be connected to a periphery of the ostomy appliance. In particular, in those embodiments where the connection(s) 60, 62 are provided as welds the connection(s) 60, 62 may be formed as part of a peripheral weld between the first, second and third walls 12, 14, 30.

The connection(s) 60, 62 may extend inwardly from a periphery of the ostomy appliance 10. Such a construction may be advantageous in guiding a user's fingers to the most effective place at which to pull the third wall 30 away from the first wall 12.

The connection(s) 60, 62 may be inclined with respect to the midline M described above. Again, this construction may be advantageous in guiding a user's fingers to the most effective place at which to pull the third wall 30 away from the first wall 12.

The connection(s) 60, 62 may, in hand with the second and third walls 14, 30, define a pocket for receipt of a user's fingers. The pocket may also be defined at least partly by the filter 9. In particular the pocket may be defined between the second and third walls 14, 30, the first and second connections 60, 62 and the filter 9.

In use a user may use the pocket by moving the bottom edge 38 of the third wall 30 away from the second wall 14 and extending their fingers into the pocket. In the present embodiment this would be between the first and second connections 60, 62. A user may then move their fingers away from the first wall 12 and thus move the third wall 30 in a direction away from the first wall 12 as described above.

In the present embodiment the second discrete area W of each connection 60, 62 is less than the first discrete area F of the filter 9. In particular the combined area W+W defined by the connections 60, 62 is less than the area F defined by the filter 9. However, in other embodiments the area W defined by the or each connection 60, 62 may be the same as the area F defined by the filter 9 or may be greater than the area F defined by the filter 9, either separately or in combination.

The lower end 13 of the appliance 10 may be provided with an outlet 22 including an outlet wall (as shown in dashed lines in FIGS. 1 to 3 and as shown in FIG. 6). The outlet 22 may be formed by portions of the first and second walls 12, 14, again, as is well known in the art. In other words the outlet 22 may be a continuation of one or both of the first and second walls 12, 14 which define the waste collecting cavity 18. In other embodiments the outlet 22 may be formed from a wall which is separate to the first and second walls 12, 14. Such an outlet 22 may be connected to one of the first and/or second walls which define the waste collecting cavity 18.

The outlet 22 may extend downwardly away from the stoma-receiving opening 16 and terminate at an opening 24. The opening 24 may permit waste collected in the collecting cavity 18 to be expelled from the appliance 10. The opening 24 may be movable between a first, closed, condition and a second, open, condition. When the opening 24 is in the second, open, condition waste may be permitted to move from the waste collecting cavity 18 through the outlet 22 and out of the opening 24 to exit the ostomy appliance 10.

To assist a user in moving the outlet opening from the first condition to the second condition the ostomy appliance 10 may include a user graspable member, or tab, 40 which is positioned at or near the outlet opening 24 and is attached to the outlet wall 22 by an attachment portion 44. The attachment portion 44 may be adhered or welded to the outlet wall 22.

The user graspable member 40 may also include a graspable portion 42 which is connected to the attachment portion 44 by a connection portion 46. A user may operate the user graspable member 40 by grasping the graspable portion 42 and moving the graspable portion 42 in a direction away from the outlet 22 and a user.

As shown in the figures, first and second elongate stiffening members 26a, 26b may be provided to assist with opening, closing and sealing of the opening 24. However, in other embodiments only a single stiffening member 26a may be provided. The stiffening member 26a may be positioned at or near the opening 24.

The first stiffening member 26a may be connected, e.g. adhered or welded, to the exterior surface of the first wall 12, which faces the user and is positioned at or near the outlet opening 24. In alternative embodiments the first stiffening member 26a forms part of the outlet 22. The first stiffening member 26a may extend laterally across the outlet 22 such that it extends generally perpendicularly to the direction in which waste exits the appliance 10, as shown in FIG. 3.

The second stiffening member 26b may be positioned adjacent and above the first stiffening member 26a, further away from the opening 24 of the outlet 22 than the first stiffening member 26a, and is connected, e.g. adhered or welded, to the exterior surface of the first wall 12 (the same wall as the first stiffening member 26a). The second stiffening member 26b may also extend laterally across the outlet 22 such that it extends generally perpendicularly to the direction in which waste exits the appliance 10, as shown in FIG. 2. The stiffening members 26a, 26b may be substantially the same.

The outlet 22, when present, may be foldable, or rollable, between an extended position and a stowed position. The ostomy appliance 10 may include a security flap 27 (which may lie underneath the comfort layer 17) to secure the outlet in its stowed position. In the stowed position the attachment portion 44 and graspable portion 42 may lie in generally the same plane.

The outlet 22 may be stored, or stowed, by being rolled up around the stiffening members 26a, 26b, as is generally known in the art. The stiffening members 26a, 26b may define fold lines L1, L2 which the outlet 22 folds about.

The stiffening members 26a, 26b may in particular be rolled up two or three times, to seal the opening 24 in a closed condition. Any suitable fastening means, e.g. hook, and loop (Velcro) or adhesive, may be provided on respective faces of the flap 27 and outlet 22 to secure the outlet 22.

A second embodiment of the invention will now be described. Features shared in common with the first embodiment of the invention will be provided with the same reference numerals with the addition of 100. Only the differences between the first and second embodiments will be described.

FIG. 8 shows a second embodiment of the invention. In this embodiment of the invention the connections 160, 162 between the second and third walls 14, 30 are provided as obrounds of adhesive. By this we mean that each connection 160, 162 has first and second generally parallel sides 170, 172 which are connected by third and fourth generally curved sides 174, 176. The third and fourth sides 174, 176, in particular, are semi-circular.

The first and second sides 170, 172 are inclined to the midline M and/or a generally vertical axis of the ostomy appliance. In particular the connection(s) 160, 162 each have a generally elongate axis A, with which the first and second sides 170, 172 may be parallel, which is inclined to a generally vertical axis of the ostomy appliance 110. The generally elongate axis A and/or the first and second sides 170, 172 may be inclined to the midline M at an angle of roughly 45 degrees. However, other angles may be used without departing from the scope of the invention. The elongate axis A of the or each connection 160, 162 intersects the gas vent 8 and the filter 9. In other embodiments the elongate axes A of the connections 160, 162 intersect each other below the gas vent 8 and/or filter 9. In further envisaged embodiments the elongate axes A of the connections 160, 162 may intersect each other above the gas vent 8 and/or filter 9. As in the first embodiment the first and second connections 160, 162 are between the second and third walls, 114, 130 and are mirror images of each other.

A third embodiment of the invention will now be described. Features shared in common with the first embodiment of the invention will be provided with the same reference numerals with the addition of 200. Only the differences between the first and second embodiments will be described.

FIGS. 9 and 10 show a third embodiment of the invention. In the third embodiment the gas vent 208 is provided in the first wall 212 for venting gas from the collecting cavity 218 to atmosphere. The filter 209 is positioned over the gas vent 208 on an outwardly facing surface 252 of the first wall 212. In other embodiments the filter 209 may be positioned over the gas vent 208 on an inwardly facing surface 232 of the first wall 212.

Operation of the ostomy appliance 210 is similar to the ostomy appliance 10 and 110 and so will not be described here again.

Representative features are set out in the following clauses, which stand alone or may be combined, in any combination, with one or more features disclosed in the text and/or drawings of the specification.

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

Although certain example embodiments of the invention have been described, the scope of the appended claims is not intended to be limited solely to these embodiments. The claims are to be construed literally, purposively, and/or to encompass equivalents.

1. An ostomy appliance comprising:
first and second walls connected to each other;
a stoma-receiving opening for receipt of a stoma;
a collecting cavity defined between the first and second walls wherein the stoma-receiving opening is fluidly connected to the collecting cavity;
a connection member connected to the first wall for attaching the appliance to a user;
a gas vent for venting gas from the collecting cavity to atmosphere;
a filter positioned over the gas vent, said filter defining a first discrete area;
a third wall optionally connected to the first and/or second wall;
wherein the second wall includes a first surface and the third wall includes a second surface;
wherein the first and second surfaces face each other and are connected at a connection; and
wherein the connection defines a second discrete area.

2. An ostomy appliance according to claim 1, wherein the first wall, the second wall and the third wall are configured such that movement of the third wall away from the first wall at least partially separates the first and second walls so as to permit air to flow through the gas vent into the collecting cavity.

3. An ostomy appliance according to claim 1, wherein the third wall overlies the filter.

4. An ostomy appliance comprising:
first and second walls connected to each other;
a stoma-receiving opening for receipt of a stoma;
a collecting cavity defined between the first and second walls wherein the stoma-receiving opening is fluidly connected to the collecting cavity;
a connection member connected to the first wall for attaching the appliance to a user;
a gas vent provided in the first wall for venting gas from the collecting cavity to atmosphere;
a filter positioned over the gas vent; and
a third wall connected to the second wall at a connection;
wherein the first wall, the second wall and the third wall are configured such that movement of the third wall in a direction away from the first wall at least partially separates the first and second walls and permits air to flow through the gas vent into the collecting cavity.

5. An ostomy appliance according to claim 4 wherein the filter defines a first discrete area; and
wherein the connection defines a second discrete area.

6. An ostomy appliance according to claim 1, wherein the third wall includes a comfort layer.

7. An ostomy appliance according to claim 1, further comprising a plurality of connections between the second and third walls.

8. An ostomy appliance according to claim 1, wherein the connection between the second and third walls is an adhesive connection.

9. An ostomy appliance according to claim 1, wherein the connection between second and third walls is provided by a weld.

10. An ostomy appliance according to claim 1, wherein the connection is positioned vertically below the filter, in an in use position of the appliance.

11. An ostomy appliance according to claim 1, wherein the connection is positioned at least partially above a generally horizontal line which extends through the stoma-receiving opening.

12. An ostomy appliance according to claim 1, wherein the connection has a generally elongate axis which is inclined to a generally vertical axis of the ostomy appliance.

13. An ostomy appliance according to claim 1, wherein the connection has a generally elongate axis which intersects the filter and/or gas vent.

14. An ostomy appliance according to on claim 7, further comprising first and second connections between the second and third walls where the first and second connections are mirror images of each other.

15. An ostomy appliance according to claim 1, wherein the third wall is positioned in an upper portion of the appliance and is connected to the second wall of the appliance.

16. An ostomy appliance according to claim 1, wherein the third wall covers a top portion of the appliance and extends downwardly at least past a main horizontal axis of the stoma-receiving opening.
